(19) United States
(12) Patent Application Publication
(10) Pub. No.: US 2014/0341813 A1
(43) Pub. Date: Nov. 20, 2014

(54) INDOCYANINE GREEN-CONTAINING PARTICLE AND METHOD OF PRODUCING THE PARTICLE

(71) Applicant: CANON KABUSHIKI KAISHA, Tokyo (JP)

(72) Inventor: Yoshinori Tomida, Atsugi-shi (JP)

(73) Assignee: CANON KABUSHIKI KAISHA, Tokyo (JP)

(21) Appl. No.: 14/260,347

(22) Filed: Apr. 24, 2014

(30) Foreign Application Priority Data
May 17, 2013 (JP) 2013-105265

(51) Int. Cl. A61K 49/22; A61K 49/00

(52) U.S. Cl. CPC A61K 49/227; A61K 49/0084
USPC 424/9.44; 424/9.6

(57) ABSTRACT

To encapsulate indocyanine green (ICG) at a high concentration in a liposome without causing the agglomeration of ICG so that ICG remains as a monomer in an ICG-containing particle to be used as, for example, a contrast agent for fluorescence imaging or photoacoustic imaging, provided is a method of producing an indocyanine green-containing particle, including the step of mixing indocyanine green, a particle, and a solution containing 1 mM or more and 10 M or less of a chaotropic agent.

### BACKGROUND OF THE INVENTION

1. Field of the Invention

The present invention relates to a particle containing indocyanine green and a method of producing the particle.

2. Description of the Related Art

In recent years, a fluorescence imaging method or a photoacoustic imaging method has attracted attention as an imaging method that allows non-invasive diagnosis.

The fluorescence imaging method involves irradiating a fluorescent dye with light and detecting fluorescence emitted from the dye, and is widely used in various types of imaging. The photoacoustic imaging method involves detecting an intensity and generation position of an acoustic wave resulting from volume expansion caused by heat released from a molecule as an object to be measured irradiated with light, to thereby obtain an image of the object to be measured. In the fluorescence imaging method or photoacoustic imaging method, a dye may be used as a contrast agent for increasing an intensity of fluorescence or an acoustic wave from a site to be measured.

In the contrast agent described above, in order that a signal intensity (intensity of fluorescence or an acoustic wave) may be effectively amplified, an improvement in absorption efficiency of irradiation energy by the following is desired. A dye that absorbs light to emit fluorescence or an acoustic wave is accumulated in, for example, a micelle, a polymer micelle, a liposome, or any other particle (when the term “particle” is simply used, hereinafter, the term means a generic term for the foregoing unless otherwise stated) to increase a dye density.

Indocyanine green (hereinafter sometimes abbreviated as ICG) is known as a dye known to emit fluorescence or an acoustic wave through light absorption. It should be noted that the ICG as used herein refers to a compound having a cyanine skeleton and having a structure represented by the following chemical formula 1.

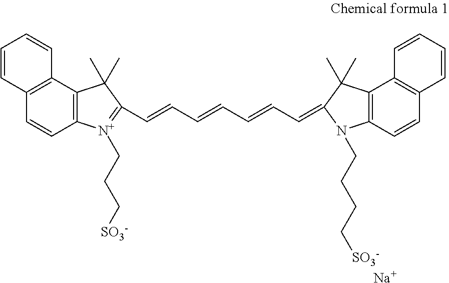

In this regard, however, H⁺ or K⁺ as well as Na⁺ may be used as a counter ion. In addition, sodium iodide NaI may be added.

However, ICG has a low molecular weight, and hence has a small size and low retentivity in a lymph node. Accordingly, a contrast agent for a lymph node having a layer size has been desired. As a particle containing ICG and having a larger size, “Enhanced photo-stability, thermal-stability and aqueous-stability of indocyanine green in polymeric nanoparticulate systems”, Journal of Photochemistry and Photobiology B: Biology, 74, 29-38 (2004) discloses an ICG-containing poly(lactide-co-glycolide) (hereinafter, sometimes abbreviated as PLGA) particle obtained by an emulsion solvent diffusion method using polyvinyl alcohol (PVA) as a surfactant.

However, the ICG-containing particle disclosed in Journal of Photochemistry and Photobiology B: Biology, 74, 29-38 (2004) has a limit in terms of the amount of a dye that can be loaded into a liposome, the ICG content of a contrast agent particle is low, the amount of ICG to be transported to a target tissue reduces, and contrast sensitivity becomes insufficient. As a result, there arises a need for the administration of a large amount of ICG-containing particles, which causes a problem in that an excessive burden is imposed on a patient.

In a contrast agent containing an ICG-containing particle, the ICG content of the particle in a living organism needs to be excellent. However, a general liposome production method has a limit in terms of the amount of a dye that can be loaded into a liposome. Specifically, in a hydration dispersion method, a liposome encapsulating an ICG aqueous solution can be formed by subjecting a phospholipid film after dissolution in an organic solvent and the removal of the solvent to ultrasonic stirring together with the ICG aqueous solution. In such method, the volume of an internal aqueous phase calculated from the number of liposomes determined from the volume and dry weight of, for example, a liposome having a particle size around 70 nm is only around 0.5 vol % of the entirety.

Even when an ICG aqueous solution whose concentration is close to a saturated concentration is subjected to hydration dispersion and concentrated by ultrafiltration, centrifugation, or the like, the ICG molar absorption coefficient ε of the entire contrast agent is of the order of 10⁸.

In a Bangham method as another liposome production method, a liposome containing ICG in a phospholipid can be formed by: mixing and dissolving a methanol solution of ICG in a chloroform solution of the phospholipid; removing the solvents to provide a phospholipid film containing ICG; and subjecting the film to ultrasonic stirring together with a buffer aqueous solution. In such method, the volume of a liposome lipid membrane calculated from the number of liposomes determined from the volume and dry weight of the same liposome having a particle size around 70 nm as that described above is only around 1 vol % of the entirety.

In this case as well, the ICG molar absorption coefficient ε of the entire contrast agent is of the order of 10⁸. Accordingly, an ICG-containing particle having a high ICG content has been demanded.

In view of the foregoing, the present invention provides a method of producing a particle based on a pH-gradient method by which ICG can be transported from an external aqueous phase to an internal aqueous phase to increase the concentration of ICG in the internal aqueous phase.

It has been known that in each of the conventional liposome production methods including the pH-gradient method, when a high-concentration ICG aqueous solution (1.5 mM or more according to “J-aggregation and disaggregation of indocyanine green in water”, Chemical Physics, Volume 220, 385-392 (1997)) is brought into a state of being heated to a temperature equal to or more than 60° C. as the phase transition temperature of a phospholipid (20° C., 45° C., 80° C., or 90° C. with reference to 65° C. in Chemical Physics, Volume 220, 385-392 (1997)), the formation of a J-aggregate (hereinafter sometimes abbreviated as “J-aggregation”) starts and the nucleation of the aggregate promotes further J-aggregation.

However, in the pH-gradient method, an encapsulation ratio tends to be higher as the concentration of ICG in an external aqueous phase increases. Accordingly, with a view to achieving an encapsulation ratio higher than that of the pH-gradient method, setting the concentration to 1.5 mM or more as described in Chemical Physics, Volume 220, 385-392 (1997) has been generally performed, and hence the formation of a J-aggregate at the time of a heat treatment for encapsulation has been inevitable. Once ICG forms a J-aggregate, it becomes difficult to increase an ICG encapsulation ratio in a particle.

This is because of the following reason. The pH-gradient method is also called trans-membrane gradient and is such that a nonionic solute in an external aqueous phase transfers to an internal aqueous phase beyond a phospholipid membrane. Accordingly, at the time of the transfer, a transfer amount increases as a molecular volume reduces. In addition, as described in “Degree of aggregation of indocyanine green in aqueous solutions determined by Mie Scattering”, Chemical Physics, Volume 220, 373-384 (1997), the J-aggregate of ICG is known to be an aggregate having 20,000 to 50,000 molecules and is too large to transfer through the phospholipid membrane.

An object of the present invention is to encapsulate a large amount of ICG in a particle while causing ICG to remain as a monomer.

### SUMMARY OF THE INVENTION

There is provided a method of producing an indocyanine green-containing particle (ICG-containing particle), including mixing ICG and a particle in a solution containing 1 mM or more and 10 M or less of a chaotropic agent.

Further features of the present invention will become apparent from the following description of exemplary embodiments with reference to the attached drawings.

### BRIEF DESCRIPTION OF THE DRAWINGS

FIG. 1 illustrates an example of a particle in one embodiment of the present invention.

FIG. 2 illustrates an example of another particle in one embodiment of the present invention.

FIG. 3 shows the absorption spectra of an ICG aqueous solution and the J-aggregate of ICG.

FIG. 4 shows the list of the results of the measurement of the spectral shifts of an aqueous solution containing 1.5 mM of indocyanine green (ICG, manufactured by Nihon Koteisho Kyokai) and urea of the present invention in Example 1 before and after heating at 60° C. for 30 minutes, the measurement being performed at respective urea concentrations of 100 mM or less. A square at the left end shows the absorption spectrum of only a 100 mM urea aqueous solution free of ICG. The axis of abscissa of each square indicates a wavelength, and values at its left and right ends are 500 nm and 950 nm, respectively, and the axis of ordinate thereof indicates an absorbance and its scale is arbitrary. The upper stage of the two vertical squares shows an absorption spectrum before the heating and the lower stage thereof shows an absorption spectrum after the heating. The square at the left end corresponds to a blank free of ICG, a second square from the left end shows the absorption spectrum of a solution obtained by adding an additive having a urea concentration of 100 mM or less (the urea concentration is described in the upper portion of the list) to 1.5 mM of ICG, and the upper stage and the lower stage show spectra before and after the heating, respectively.

FIG. 5 shows the list of the results of the measurement of the spectral shifts of an aqueous solution containing 1.5 mM of indocyanine green (ICG, manufactured by Nihon Koteisho Kyokai) and citric acid of the present invention in Example 1 before and after heating at 60° C. for 30 minutes, the measurement being performed at respective citric acid concentrations of 100 mM or less. A square at the left end shows the absorption spectrum of only a 100 mM citric acid aqueous solution free of ICG. The axis of abscissa of each square indicates a wavelength, and values at its left and right ends are 500 nm and 950 nm, respectively, and the axis of ordinate thereof indicates an absorbance and its scale is arbitrary. The upper stage of the two vertical squares shows an absorption spectrum before the heating and the lower stage thereof shows an absorption spectrum after the heating. The square at the left end corresponds to a blank free of ICG, a second square from the left end shows the absorption spectrum of a solution obtained by adding an additive having a citric acid concentration of 100 mM or less (the citric acid concentration is described in the upper portion of the list) to 1.5 mM of ICG, and the upper stage and the lower stage show spectra before and after the heating, respectively.

FIG. 6 shows the list of the results of the measurement of the spectral shifts of an aqueous solution containing 1.5 mM or less of indocyanine green (ICG, manufactured by Nihon Koteisho Kyokai) before and after heating at 60° C. for 30 minutes. A square at the left end shows the absorption spectrum of only water free of ICG. The axis of abscissa of each square indicates a wavelength, and values at its left and right ends are 500 nm and 950 nm, respectively, and the axis of ordinate thereof indicates an absorbance and its scale is arbitrary. The upper stage of the two vertical squares shows an absorption spectrum before the heating and the lower stage thereof shows an absorption spectrum after the heating. The square at the left end corresponds to a blank free of ICG, a second square from the left end shows the absorption spectrum of a 1.5 mM ICG aqueous solution (the ICG concentration is described in the upper portion of the list), and the upper stage and the lower stage show spectra before and after the heating, respectively.

FIG. 7 shows the absorption spectrum of an ICG-containing particle obtained by a urea-added pH-gradient method in Example 2 of the present invention. An absorption peak is observed at around 780 nm.

FIG. 8 shows the absorption spectrum of an ICG-containing particle obtained by a conventional pH-gradient method in which urea is not added in Comparative Example 1 of the present invention. An absorption peak is observed at around 895 nm.

FIG. 9 shows the absorption spectrum of an ICG-containing particle obtained without the provision of any pH gradient in Comparative Example 2 of the present invention. An absorption peak is observed at around 780 nm.

FIG. 10 shows data on comparison between the particle sizes of a liposome obtained in Example 2 of the present invention before the encapsulation of ICG with a pH gradient, and after the encapsulation and purification.

### DESCRIPTION OF THE EMBODIMENTS

Hereinafter, embodiments of the present invention are described but the present invention is not limited to these embodiments.

According to a first embodiment of the present invention, there is provided a method of producing an ICG-containing particle, including the step of mixing ICG and a particle in a solution containing 1 mM or more and 10 M or less of a chaotropic agent.

In addition, in the method of producing an ICG-containing particle according to this embodiment, the solution containing the chaotropic agent preferably contains 6.25 mM or more of the chaotropic agent and more preferably contains 100 mM or less of the chaotropic agent.

In addition, in the step of mixing ICG and the particle in the solution containing 1 mM or more and 10 M or less of the chaotropic agent, a solution containing the chaotropic agent in which ICG is dissolved and the particle are preferably mixed. According to this embodiment, in, for example, a pH-gradient method involving encapsulating ICG from the external aqueous phase of a liposome in its internal aqueous phase, the J-aggregation of ICG can be suppressed and a particle having a high ICG encapsulation ratio can be produced by adding a chaotropic agent such as urea to the external aqueous phase.

The chaotropic agent refers to a substance that reduces an interaction between water molecules to destabilize a structure based on the interaction. Preferred examples of the chaotropic agent can include urea, guanidine, iodine, and ions thereof. A preferred concentration of the chaotropic agent is 1 mM or more and 10 M or less. In the case of urea, a particularly preferred concentration can be, for example, 10 mM or more and 1 M or less.

The solution containing 1 mM or more and 10 M or less of the chaotropic agent preferably has a pH of less than 7 and a more preferred value for the pH can be, for example, less than 5. It should be noted that the solution is not particularly limited and refers to, for example, water, an aqueous solvent, a buffer solution, and a particle dispersion medium, and examples thereof can include a solution A, a solution B, and a solution containing 100 mM of urea and having a pH of 3.0 in Table 2.

In addition, in the present invention, the particle is not particularly limited but a liposome can be given as a preferred example thereof.

According to a preferred embodiment of the present invention, there is provided an indocyanine green-containing particle containing indocyanine green and a particle, the indocyanine green-containing particle further containing a chaotropic agent.

In this embodiment, a ratio “O.D. (895 nm)/O.D. (780 nm)” of the absorbance of the particle for light having a wavelength of 895 nm (derived from a J-aggregate) to its absorbance for light having a wavelength of 780 nm (derived from a monomer) is preferably 1.0 or less, but the ratio is not particularly limited, and an abundance ratio between the J-aggregate and monomer in the particle is not particularly limited.

Similarly, a ratio “O.D. (700 nm)/O.D. (780 nm)” of the absorbance of the particle for light having a wavelength of 700 nm (derived from an H-aggregate) to its absorbance for the light having a wavelength of 780 nm (derived from the monomer) is preferably 1.0 or less, but the ratio is not particularly limited, and an abundance ratio between the H-aggregate and monomer in the particle is not particularly limited.

The particle in this embodiment can further contain a phospholipid. The phospholipid of the particle in this embodiment more preferably forms a double membrane and the phospholipid preferably accounts for 30 wt % or more of the particle. In addition, the phospholipid can be, for example, a lipid having a phosphoric acid diester bond. The particle of this embodiment can further contain cholesterol and a liposome can be given as a preferred example of the particle. Although the particle size of the particle is not limited, a preferred example thereof can be as follows: its average particle size is 1,000 nm or less, more preferably 200 nm or less. The particle size can be measured by, for example, a light scattering method.

In addition, the particle of this embodiment can further have a PEG chain on its surface.

A preferred embodiment of the present invention is a photoacoustic imaging contrast agent containing the ICG-containing particle. The photoacoustic imaging contrast agent of this embodiment preferably contains the ICG-containing particle and a dispersion medium for dispersing the ICG-containing particle. In this case, the contrast agent can contain a dispersion medium containing the chaotropic agent.

(J-Aggregate)

ICG is known to form a J-aggregate under a specific condition (Chemical Physics, Volume 220, 385-392 (1997), Chemical Physics, Volume 220, 373-384 (1997), and “Microstructure of indocyanine green J-aggregates in aqueous solution”, Chemical Physics, Volume 269, 399-409 (2001)). The J-aggregate is a multimer having an average particle size of several micrometers, and it is known that its absorption maximum wavelength largely shifts to longer wavelengths and its absorption band becomes sharp as compared with a monomer.

The J-aggregate of ICG is defined as an aggregate having a local maximum of an absorbance between 880 nm and 910 nm as a result of the shift of an absorption wavelength as compared with the monomer out of the aggregates as the multimer structural products of ICG. It should be noted that when ICG is contained in the particle herein, the J-aggregate or H-aggregate of ICG may be contained in the particle.

Although none of the J-aggregate and the H-aggregate inhibits contrasting, the formation of any such aggregate is preferably suppressed in terms of sensitivity because aggregation reduces the absorption of light having a wavelength of 780 nm derived from an ICG monomer.

The use of the J-aggregate of ICG reduces a light source output and reduces the sensitivity because its absorption wavelength falls within the boundary wavelength region of measurement in a general fluorescence imaging apparatus or photoacoustic imaging apparatus.

In the present invention, the accumulation of ICG in a site to be measured such as a lymph node can be improved by suppressing an aggregated state and encapsulating the monomer at a high concentration.

(Particle)

In the specification of the present application, the particle is not particularly limited and refers to all particles irrespective of whether ICG is contained; the particle refers, in particular, to a particle containing ICG and is sometimes referred to as, for example, “indocyanine green-containing particle” or “ICG-containing particle.” The shape of the particle is not limited and comprehends a micelle, a polymer micelle, a liposome, and all other particles.

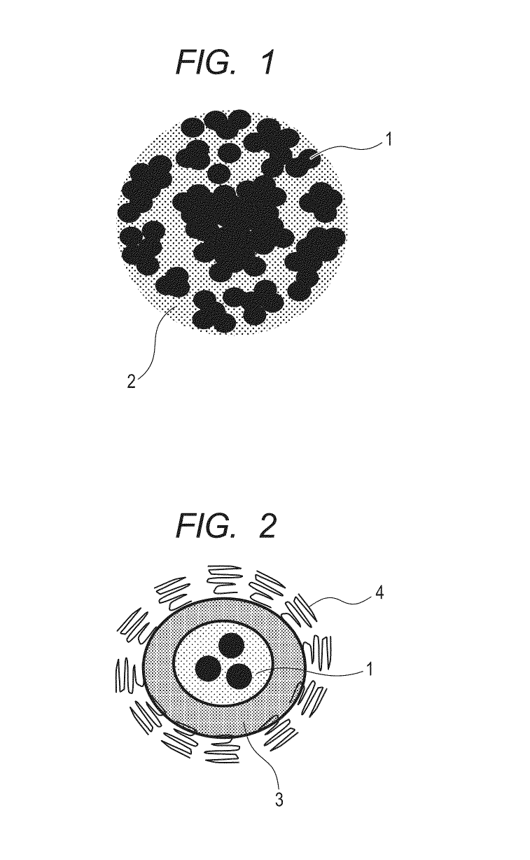

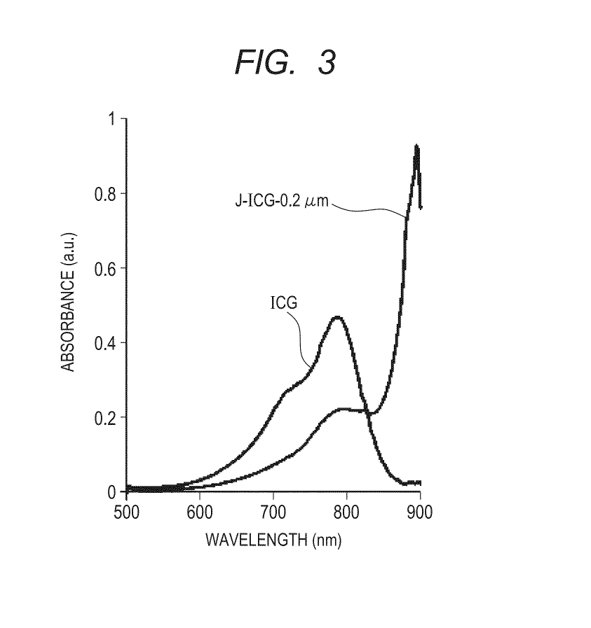

The particle of the present invention may contain an additive such as a lipid having a positively charged site in addition to ICG, or may be a particle free of any additive. The particle is not particularly limited and may be, for example, a micelle, a polymer micelle, or a liposome. In addition, a surfactant may be present on the surface of the particle. Examples of such particle include such a particle formed of an ICG 1 and a phospholipid 2 as an additive as illustrated in FIG. 1, and such a particle containing a surfactant 4 on the surface of a liposome 3 containing the ICG 1 as illustrated in FIG. 2.

(Average Particle Size)

The average particle size of the particle according to this embodiment means a hydrodynamic average particle size measured by a light scattering method. The average particle size is hereinafter sometimes simply referred to as “particle size.” The range of the particle size of the particle according to this embodiment is not particularly limited; provided that when the particle is used as a contrast agent, in particular, a contrast agent for a lymph node, setting the hydrodynamic average particle size to 1,000 nm or less can improve the ease with which the particle is taken in a lymph duct or a tissue (tissue permeability) and its retentivity in a lymph node or the tissue.

When the particle size is 1,000 nm or less, a larger amount of particles can be accumulated in a tumor site than that in a normal site in a living organism by an enhanced permeability and retention (EPR) effect. The tumor site can be specifically imaged by detecting the accumulated particles with various image-forming modalities such as fluorescence and photoacoustics. In addition, when the particle size exceeds 1,000 nm, efficient intake in a tissue such as a lymph duct cannot be expected. Consequently, the particle size is preferably set to 10 nm or more and 1,000 nm or less. The particle size is more preferably 20 nm or more and 500 nm or less, still more preferably 20 nm or more and 200 nm or less. This is because when the particle size of the particle is 200 nm or less, the particle is hardly taken in a macrophage in blood and hence its retentivity in the blood may improve.

The particle size can be measured through observation with an electron microscope or by a particle size-measuring method based on a dynamic light scattering method. When the particle size is measured based on the dynamic light scattering method, a hydrodynamic diameter is measured with a dynamic light scattering analyzer (DLS-8000, manufactured by Otsuka Electronics Co., Ltd.) by the dynamic light scattering (DLS) method.

A particle having such preferred size can be produced by filtering the aggregate with a pore filter to be described later, by employing a nanoemulsion method to be described later, or by incorporating the aggregate into a particle such as a liposome.

(Chaotropic Agent)

Examples of the chaotropic agent include urea, guanidine, iodine, and ions thereof. Of those, urea can be given as a particularly preferred example thereof. The additive in the particle may remain because the additive is used at the time of an encapsulation treatment based on the pH-gradient method in a production process. However, urea has a track record as an additive for vitamin intravenous injection (trade name: Vitalfa Injection, Conbelbe Injection, or the like) (added in an amount of 50 mg to 10 ml of an injection). In addition, according to the material safety data sheet (MSDS) of urea, the LD50 of its intravenous injection is around 5,000 mg/kg for a rat or a mouse. Accordingly, even when urea is added at 1 M as a preferred concentration range upper limit of the present invention, no toxicity is observed from the molecular weight of urea and the liquid amount of the intravenous injection.

Accordingly, in the present invention, there is no need to eliminate urea after production, and when urea remains or is added to an external aqueous phase, stability over time, i.e., a suppressing effect on the shift of the absorption wavelength of ICG can be sustained.

(Lipid Having Positively Charged Site)

The particle according to the present invention can contain, for example, a lipid having a positively charged site. Although ICG is a hydrophilic dye having a sulfonic group as a hydrophilic site, the addition of the lipid having a positively charged site results in the aggregation of the positively charged site which the additive has to the hydrophilic site of ICG, and hence can improve the hydrophobicity of ICG. Consequently, it is assumed that ICG can be solubilized in an organic solvent such as chloroform or dichloromethane.

The lipid having a positively charged site refers to a lipid having a partial structure of a cation in a part of its structure. Examples of such lipid may include: glycerolipids such as a phosphatidylcholine, a phosphatidylethanolamine, and a phosphatidylserine; sphingolipids such as a sphingomyelin, a sphingophospholipid, and sphingosine; a glycolipid such as a sphingoglycolipid having an aminosugar moiety such as neuraminic acid; synthetic cholesterols such as cholesteryl-3β-carboxyamidoethylene-N-hydroxyethylamine and 3-([N—N′,N′-dimethylaminoethane)-carbamoyl]cholesterol; synthetic lipids such as laurylamine, stearylamine, N-[1-(2,3-dioleyloxy)propyl]-N,N,N-trimethylammonium chloride (abbreviation: DOTMA), and 2,3-dioleyloxy-N-[2-(sperminecarboxyamido)ethyl]-N,N-dimethyl-1-propanaminium trifluoroacetate (abbreviation: DOSPA); and an ether-type phospholipid and a cationic lipid.

In addition, examples of the phosphatidylcholine, phosphatidylethanolamine, and phosphatidylserine include diacylphosphatidylcholine, diacylphosphatidylethanolamine, and diacylphosphatidylserine.

In addition, the lipid having a positively charged site can further have a phosphodiester bond. Examples thereof include 1,2-distearoyl-sn-glycero-3-phosphoethanolamine (DSPE), 1,2-dipalmitoyl-sn-glycero-3-phosphoethanolamine (DPPE), 1,2-dimyristoyl-sn-glycero-3-phosphoethanolamine (DMPE), 1,2-dilauroyl-sn-glycero-3-phosphoethanolamine (DLPE), 1,2-dioleoyl-sn-glycero-3-phosphoethanolamine (DOPE), 1,2-dilinoleoyl-sn-glycero-3-phosphoethanolamine (DLoPE), 1,2-dierucoyl-sn-glycero-3-phosphoethanolamine (DEPE), 1,2-distearoyl-sn-glycero-3-phospho-L-serine (DSPS), 1,2-dipalmitoyl-sn-glycero-3-phospho-L-serine (DPPS), 1,2-dimyristoyl-sn-glycero-3-phospho-L-serine (DMPS), 1,2-dioleoyl-sn-glycero-3-phospho-L-serine (DOPS), 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dilauroyl-sn-glycero-3-phosphocholine (DLPC), 1,2-dioleoyl-sn-glycero-3-phosphocholine (DOPC), and 1,2-dilinoleoyl-sn-glycero-3-phosphocholine (DLoPC).

As the lipid having a positively charged site in this embodiment, there may also be used, for example, 1,2-di-o-acyl-sn-glycero-3-phosphocholine, 1,2-diacyl-3-trimethylammonium propane chloride, o,o′-ditetradecanoyl-N-(α-trimethylammonioacetyl)diethanolamine chloride, and a hydrogenated soy phosphatidylcholine (sometimes abbreviated as HSPC).

Particularly preferred examples of the lipid having a positively charged site according to the present invention include dioleyl phosphatidyl ethanolamine and distearoyl phosphatidylcholine.

(Liposome)

The particle of the present invention can adopt the form of a liposome. The liposome as used herein means a monolayer liposome and a multilayer liposome constituted of a lipid, a glycolipid, a phospholipid, a sterol, and a combination thereof. The liposome may be constituted of a mixture of different lipids, and a derivative of a lipid such as a polyethylene glycol-bonded phospholipid can be used. A conventionally known method can be employed as a method of preparing the liposome and a liposome having desired physical properties can be obtained by appropriately selecting a method. The kind, amount, and the like of a lipid can be appropriately selected according to the applications of the liposome. The particle size and surface potential of the liposome can be controlled by taking, for example, the amount and ratio of a lipid, and the charge of the lipid into consideration.

Examples of the neutral phospholipid to be contained in the liposome include a soy or yolk lecithin, a lysolecithin, and a derivative of a hydrogenated product or hydroxide thereof. In addition, examples thereof may also include a semisynthetic phosphatidylcholine, a phosphatidylserine (PS), a phosphatidylethanolamine, a phosphatidylglycerol (PG), a phosphatidylinositol (PI), and a sphingomyelin. In addition, an alkyl or alkenyl derivative of synthetic phosphatidic acid (PA) or the like may also be used, and examples thereof include distearoylphosphatidylcholine (DSPC), dimyristoylphosphatidylcholine (DMPC), dipalmitoylphosphatidylcholine (DPPC), dioleylphosphatidylcholine (DOPC), distearoylphosphatidylserine (DSPS), distearoylphosphatidylglycerol (DSPG), and dipalmitoylphosphatidic acid (DPPA).

Examples of the glycolipid to be contained in the liposome include: a glycerolipid such as digalactosyldiglyceride; and sphingoglycolipids such as a galactosylceramide and a ganglioside.

When the liposome is adopted as the particle, any other molecule except for the foregoing may be added as a constituent molecule of a liposome membrane as required. Examples thereof include: a cholesterol acting as a membrane stabilizing agent; a glycol such as ethylene glycol; a saccharide such as dextran; a phosphoric acid dialkyl ester added to control a charge; and an aliphatic amine such as stearylamine.

(Containing of ICG in Particle)

The phrase “particle contains ICG” in the present invention refers to, for example, the case where the particle is a liposome and ICG is encapsulated in the liposome. ICG is a water-soluble substance and is typically encapsulated in the internal aqueous phase of the liposome. However, ICG has an affinity for a phospholipid and the multimerization of ICG molecules is liable to occur, and hence its localization to the surface of a liposome membrane or in a lipid double membrane can occur. Herein, the three cases, i.e., “encapsulation in the internal aqueous phase of the liposome,” “localization in the liposome membrane,” and “localization to the surface of the liposome” are collectively defined as the containing in the liposome.

(Surfactant)

A surfactant can be used in the production of the particle of this embodiment. As the surfactant, for example, a nonionic surfactant, an anionic surfactant, a cationic surfactant, a polymeric surfactant, or a phospholipid may be used. One kind of those surfactants may be used alone, or two or more kinds thereof may be used in combination. Examples of the nonionic surfactant to be used as the surfactant in this embodiment may include: polyoxyethylene sorbitan-based fatty acid esters such as Tween 20, Tween 40, Tween 60, Tween 80, and Tween 85; and Brij 35, Brij 58, Brij 76, Brij 98, Triton X-100, Triton X-114, Triton X-305, Triton N-101, Nonidet P-40, Igepol CO530, Igepol CO630, Igepol CO720, and Igepol CO730.

In addition, examples of the anionic surfactant to be used as the surfactant in this embodiment may include: sodium dodecyl sulfate; and a dodecylbenzenesulfonate, a decylbenzenesulfonate, an undecylbenzenesulfonate, a tridecylbenzenesulfonate, and a nonylbenzenesulfonate, and sodium, potassium, and ammonium salts thereof.

In addition, examples of the cationic surfactant to be used as the surfactant in the present invention may include cetyltrimethylammonium bromide, hexadecylpyridinium chloride, dodecyltrimethylammonium chloride, and hexadecyltrimethylammonium chloride.

In addition, examples of the polymeric surfactant to be used as the surfactant in the present invention may include polyvinyl alcohol, polyoxyethylene polyoxypropylene glycol, and gelatin. As a commercially available product of polyoxyethylene polyoxypropylene glycol, for example, Pluronic F68 (manufactured by Sigma-Aldrich Japan K.K.) and Pluronic F127 (manufactured by Sigma-Aldrich Japan K.K.) are given.

In addition, the phospholipid to be used as the surfactant in the present invention is preferably a phosphatidyl-based phospholipid having a functional group selected from a hydroxyl group, a methoxy group, an amino group, a carboxyl group, an N-hydroxysuccinimide group, and a maleimide group. In addition, the phospholipid to be used as the surfactant may contain a PEG chain.

In order that the EPR effect proposed as a principle of passive targeting to a tumor may be caused, a contrast agent is required to have high retentivity in blood. The introduction of polyethylene glycol into the particle of the present invention is extremely useful because of the following reason. When its interaction with a protein in blood is suppressed, the polyethylene glycol is hardly phagocytosed by a reticuloendothelial cell of a liver or the like and hence can improve the retentivity of the particle in the blood.

A function of the polyethylene glycol can be regulated by appropriately changing its molecular weight and its ratio of introduction into the particle. Polyethylene glycol having a molecular weight of 500 to 200,000 is preferably used and the molecular weight is particularly suitably 2,000 to 100,000. In addition, the ratio of introduction of the polyethylene glycol into the particle is 0.001 to 50 mol %, preferably 0.01 to 30 mol %, more preferably 0.1 to 10 mol % with respect to the lipid constituting the particle.

A known technique can be used as a method of introducing polyethylene glycol into the particle. A preferred example thereof is a method involving incorporating a polyethylene glycol-bonded phospholipid or the like into a phospholipid to be used for covering the particle in advance to produce the particle. An example of the polyethylene glycol-bonded phospholipid is a polyethylene glycol derivative of a phosphatidylethanolamine, such as a distearoylphosphatidylethanolamine-polyethylene glycol (DSPE-PEG).

As a phospholipid to be used for a surfactant containing a PEG chain and a functional group such as a hydroxyl group, a methoxy group, an amino group, an N-hydroxysuccinimide group, or a maleimide group, there may be given, for example, phospholipids such as: 1,2-distearoyl-sn-glycero-3-phosphoethanolamine-N-[poly(ethylene glycol)] (DSPE-PEG-OH) represented by the chemical formula 2; poly(oxy-1,2-ethanediyl), α-[7-hydroxy-7-oxido-13-oxo-10-[(1-oxooctadecyl)oxy]-6,8,12-trioxa-3-aza-7-phosphatriacont-1-yl]-ω-methoxy-(DSPE-PEG-OMe) represented by the chemical formula 3; N-(aminopropyl polyethyleneglycol)-carbamyl distearoylphosphatidylethanolamine (DSPE-PEG-NH2) represented by the chemical formula 4; 3-(N-succinimidyloxyglutaryl)aminopropyl polyethyleneglycol-carbamyl distearoylphosphatidyl-ethanolamine (DSPE-PEG-NHS) represented by the chemical formula 5; and N-(3-maleimide-1-oxopropyl)aminopropyl polyethyleneglycol-carbamyl distearoylphosphatidyl-ethanolamine (DSPE-PEG-MAL) represented by the chemical formula 6. It should be noted that in the chemical formulae 2 to 6, n represents an integer of 5 or more and 500 or less.

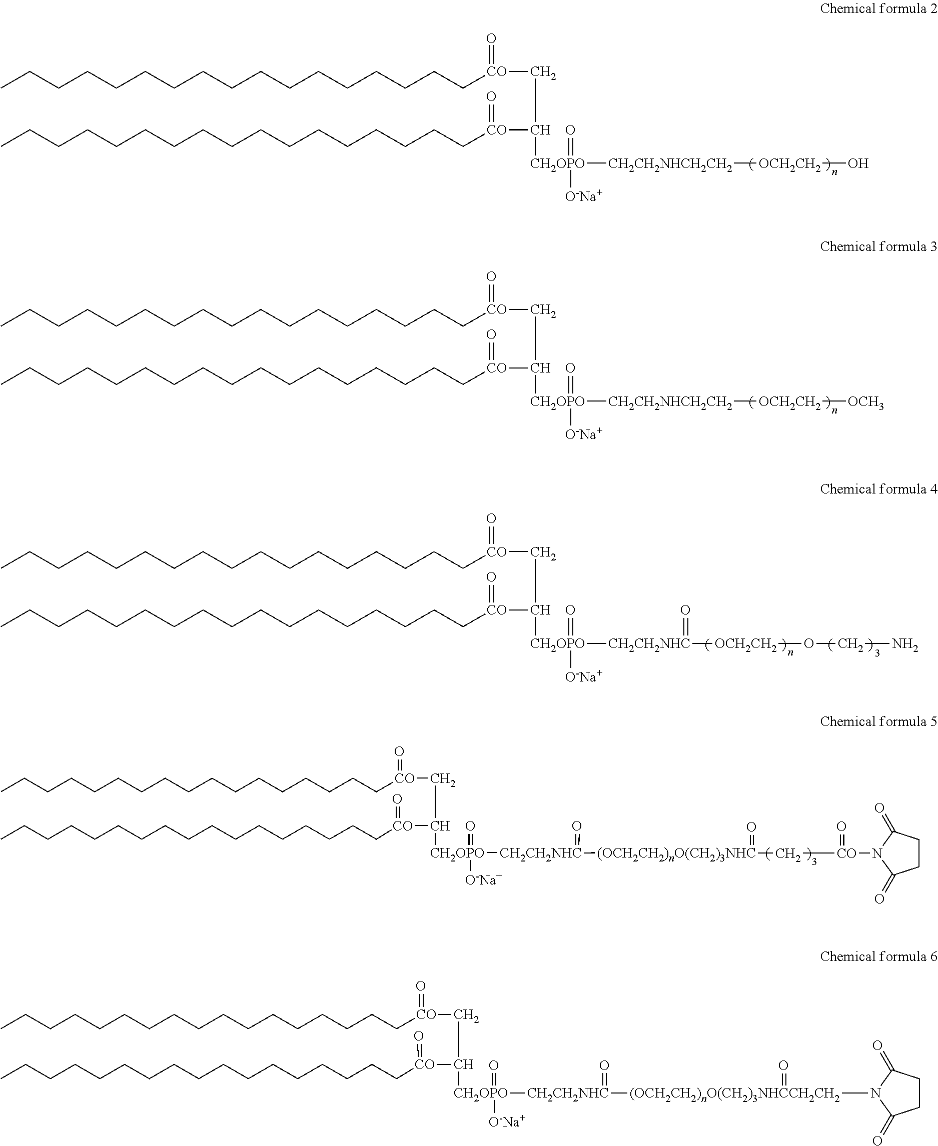

It should be noted that the number of kinds of the surfactants to be used in the present invention may be one, or two or more kinds of surfactants may be simultaneously used.

(Targeting Molecule)

In one embodiment of the present invention, a target site can be specifically labeled by immobilizing a targeting molecule to part of the particle.

The targeting molecule is, for example, a substance that specifically binds to a target site such as a tumor or a substance that specifically binds to a substance present around the target site, and can be arbitrarily selected from, for example, chemical substances such as a biomolecule and a drug. Specific examples thereof include an antibody, an antibody fragment, an enzyme, a bioactive peptide, a glycopeptide, a sugar chain, a lipid, and a molecule-recognizing compound. One kind of those substances can be used alone, or two or more kinds thereof can be used in combination. The use of the particle to which the targeting molecule is chemically bonded enables specific detection of the target site, and the tracking of the movement, localization, drug effect, metabolism, and the like of a target substance. For example, the adoption of a substance that specifically binds to a tumor as the targeting molecule enables specific detection of the tumor. In addition, the use of a substance, which specifically binds to a biological substance such as a protein or an enzyme present in a large amount around a specific disease site, as the targeting molecule enables specific detection of the disease. It should be noted that according to the particle according to this embodiment, the tumor can be detected by the EPR effect even when the particle has no targeting molecule.

(Immobilization of Targeting Molecule)

The targeting molecule may be immobilized to the particle of the present invention. Any one of the known methods can be employed as a method for the immobilization as long as the targeting molecule can be chemically bonded to the particle. For example, a method involving causing a functional group which the surfactant has and a functional group of the targeting molecule to react with each other to chemically bond the targeting molecule can be employed.

For example, when the surfactant is a phosphatidyl-based phospholipid having an N-hydroxysuccinimide group, the surfactant can be caused to react with a targeting molecule having an amino group to immobilize the targeting molecule to the particle. After immobilization of the targeting molecule, it is preferred that unreacted N-hydroxysuccinimide groups of the surfactant be inactivated through a reaction with glycine, ethanolamine, oligoethylene glycol or polyethylene glycol having an amino group at its terminal, or the like.

In addition, when the surfactant is a phosphatidyl-based phospholipid having a maleimide group, the surfactant can be caused to react with a targeting molecule having a thiol group to immobilize the targeting molecule to the particle. After immobilization of the targeting molecule, it is preferred that unreacted maleimide groups of the surfactant be inactivated through a reaction with L-cysteine, mercaptoethanol, oligoethylene glycol or polyethylene glycol having a thiol group at its terminal, or the like.

In addition, when the surfactant is a phosphatidyl-based phospholipid having an amino group, the surfactant can be caused to react with an amino group of a targeting molecule through use of glutaraldehyde to immobilize the targeting molecule to the particle. After immobilization of the targeting molecule, it is preferred that the activity of unreacted amino groups be blocked through a reaction with ethanolamine, oligoethylene glycol or polyethylene glycol having an amino group at its terminal, or the like. Alternatively, a targeting molecule may be immobilized through substitution of an amino group of the surfactant with an N-hydroxysuccinimide group or a maleimide group.

(Method of Producing Particle)

An example of a method of producing an ICG-containing particle according to this embodiment includes the steps of: preparing a particle free of ICG (empty liposome); and preparing an ICG-containing particle having ICG encapsulated therein.

(Step of Preparing Empty Liposome Particle Free of ICG)

Hereinafter, a nonlimitative example of the method of producing the particle according to this embodiment is given.

(Production Example of Empty Liposome Particle)

(Method of Preparing Liposome)

The liposome can be prepared by a known liposome production method. Examples of the known technology include a method by Bangham et al., and modifications thereof, an ultrasonic treatment method, an ethanol injection method, a cholic acid (surfactant) method, a freezing and thawing method, an antiphase evaporation method, and a method involving using a commercial kit. A liposome prepared by any one of those known methods can be used in the present invention. That is, ICG can be encapsulated at the time of, or after, the preparation of the liposome.

A preferred example of the method of producing the liposome follows the liposome production method reported by Bangham et al. That is, the liposome is formed by: dissolving and mixing raw materials for the liposome such as the phospholipid in an organic solvent; removing the organic solvent under reduced pressure to dry and harden the lipid; dispersing the dried and hardened product in an aqueous medium; and uniformizing the resultant through ultrasonic irradiation. After that, the liposome solution can be warmed or subjected to an ultrasonic treatment.

A production example of the liposome encapsulating ICG is given below. Raw materials for the liposome such as the phospholipid are dissolved and mixed in an organic solvent, and then the solutes are dried and hardened by removing the organic solvent under reduced pressure. The dried and hardened product is dispersed in a neutral buffer solution, and then the resultant is uniformized through ultrasonic irradiation to form the liposome. Thus, the liposome containing the neutral buffer solution in itself is prepared. After that, the buffer solution outside the liposome is replaced with an acidic buffer solution. Thus, a liposome dispersion liquid having such a pH gradient that the inside of the liposome is neutral and the outside thereof is acidic is prepared. A solution prepared by dissolving ICG in an acidic buffer solution is added to the resultant liposome dispersion liquid, and then the mixture is warmed and stirred at a temperature equal to or more than the transition temperature of the phospholipid as a raw material for 30 minutes. Thus, ICG that has J-aggregated is encapsulated in the liposome.

This is because ICG is J-aggregated when heated at 60° C. in a citrate buffer having a pH of 3 as the acidic buffer solution.

A method of encapsulating a drug in a liposome involving utilizing a pH gradient is effective for the encapsulation of a basic drug in a liposome. However, ICG is an acidic drug and hence the inventors of the present application tried the preparation of a liposome having a pH gradient opposite thereto. It was found that when ICG was encapsulated under the condition, most of the encapsulated ICG J-aggregated.

The J-aggregation can be described based on reaction kinetics, and a concentration, a reaction temperature, and a catalyst serve as parameters for a reaction rate.

That is, in the J-aggregation of ICG, an ICG concentration, a heating temperature, a phospholipid, citric acid, a hydrogen ion, and the like may function as positive catalysts. It should be noted that it can be assumed that urea of the present invention functions as a negative catalyst because urea suppresses the J-aggregation.

(Step of Preparing Liposome Particle Having ICG Encapsulated Therein)

The inventors of the present invention have succeeded in encapsulating ICG as a monomer at a high concentration in a liposome by further improving the known pH-gradient method.

That is, according to the method of preparing ICG, an aqueous solution of ICG that hardly J-aggregates even when heated can be prepared by adding urea at a concentration of 100 mM to an acidic buffer solution of ICG (ICG concentration=1.5 mM). The particle of the present invention is obtained by encapsulating the resultant aqueous solution of ICG as an external aqueous phase in the empty liposome obtained by the known step of preparing an empty liposome particle free of ICG after its preparation.

An encapsulation treatment involves: replacing the buffer solution outside the liposome with the acidic buffer solution containing urea of the present invention to provide a liposome dispersion liquid having such a pH gradient that the inside of the liposome is neutral and the outside thereof is acidic; adding the ICG aqueous solution to the liquid; and stirring the mixture under heating at 60° C. for 30 minutes or more.

As described above, it can be assumed that in the present invention, urea functions as a negative catalyst because urea suppresses J-aggregation.

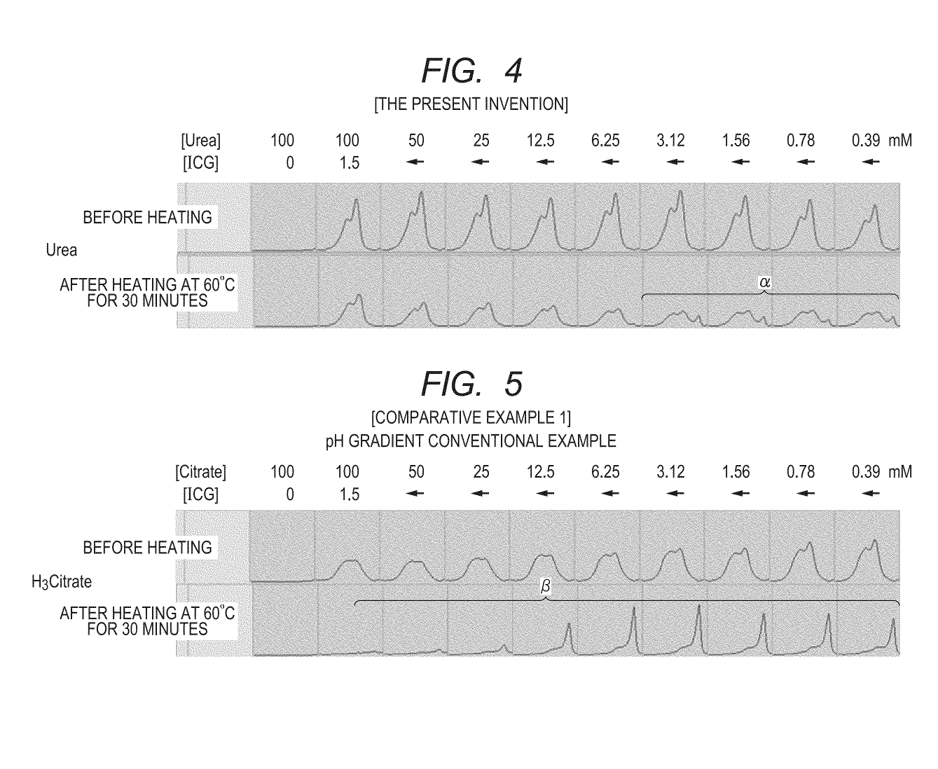

It should be noted that the effect of the addition of urea is not proportional to a urea concentration because urea functions as a catalyst. As can be seen from the experimental results of FIG. 4, the effect may be the same as long as urea is added at a certain concentration or more. The experimental results of FIG. 4 show that in the present invention, a urea concentration of 1 mM or more, in particular, 10 mM or more is preferred.

The liposome of the present invention is prepared by purifying the liposome in which ICG has been encapsulated as described above through, for example, centrifugal separation, size exclusion chromatography, or ultrafiltration. Important points in the preparation of the liposome encapsulating ICG of the present invention are the following two: an environment having a high concentration of ICG for encapsulating ICG and the phase transition of the liposome through warming.

The concentration of ICG in the solution is at least 0.1 mM or more, preferably 1.0 mM or more, and the warming is performed at not less than 20° C., preferably 37° C. or more, more preferably 65° C. or more. However, a preferred warming temperature ranges from 37° C. to 65° C. because the decomposition of the dye is promoted at 90° C.

(Contrast Agent)

The particle according to this embodiment can be used as a contrast agent for fluorescence imaging or for photoacoustic imaging because the particle contains ICG and can absorb near infrared light to emit fluorescence or an acoustic wave. In addition, the particle can be used as a contrast agent for visual detection because ICG has a green color.

Here, the “contrast agent” in the specification is mainly defined as a substance capable of causing a difference in contrast between a tissue or molecule which one wishes to observe, the tissue or molecule being present in a specimen, and a tissue or molecule around the tissue or molecule to improve the sensitivity of the detection of morphological information or positional information about the tissue or molecule which one wishes to observe. Here, the term “fluorescence imaging” or “photoacoustic imaging” means that the tissue or molecule is imaged with, for example, a fluorescence-detecting apparatus or a photoacoustic signal-detecting apparatus.

A contrast agent according to this embodiment includes the particle according to this embodiment and a dispersion medium in which the particle is dispersed. The dispersion medium is a liquid substance for dispersing the particle according to this embodiment, and examples thereof include physiological saline and distilled water for injection. In addition, the contrast agent may have a pharmacologically acceptable additive such as table salt or glucose. The contrast agent according to this embodiment may be such that the particle according to this embodiment is dispersed in the dispersion medium in advance, or may be used as described below. The particle according to this embodiment and the dispersion medium are turned into a kit, and the particle is dispersed in the dispersion medium before administration into a living organism.

(Fluorescence Imaging Method)

The contrast agent according to this embodiment may also be used for a fluorescence imaging method. A fluorescence imaging method using the contrast agent according to this embodiment includes at least the steps of: administering the contrast agent according to this embodiment to a specimen or a sample obtained from the specimen; irradiating the specimen or the sample obtained from the specimen with light; and measuring fluorescence from a substance derived from the particle present in the specimen or in the sample obtained from the specimen.

An example of the fluorescence imaging method using the contrast agent according to this embodiment is as described below. That is, the contrast agent according to this embodiment is administered to a specimen, or is added to a sample such as an organ obtained from the specimen. It should be noted that the specimen refers to all living organisms such as an experimental animal and a pet without any particular limitation. Examples of the specimen or the sample obtained from the specimen may include an organ, a tissue, a tissue section, a cell, and a cell lysate. After the administration or addition of the particle, the specimen or the like is irradiated with light in a near infrared wavelength region.

The imaging can be performed with a commercial fluorescence imaging apparatus IVIS (such as an IVIS (trademark) Lumina Imaging System) and an ICG filter.

As described above, many photoimaging apparatus are designed to use the wavelength range and the IVIS also corresponds to the absorption wavelength of an ICG monomer, i.e., 780 nm (excitation passband of a filter set 4: 705 to 780 nm).

TABLE 1
Standard Fluorescent Filter Sets

| | | Back- | | | |
|---|---|---|---|---|---|
| | | ground | Excitation | Emission | Dyes, Fluorescent |
| Filter | | Passband | Passband | Passband | Proteins, and |
| Set | Label | (nm) | (nm) | (nm) | Quantum Dots |
| 1 | Green | 410-440 | 445-490 | 515-575 | GFP, EGFP, FITC |
| 2 | Red | 460-490 | 500-550 | 575-650 | DsRed, PKR26, |
| | | | | | Qdot ™605 |
| 3 | Far-red | 580-510 | 615-655 | 695-770 | Cy5.5, Alexa |
| | | | | | Fluor ™, |
| | | | | | Qdot ™705 |
| 4 | NIR | 665-695 | 705-780 | 810-885 | ICG, |
| | | | | | Qdot ™800 |

In addition, it has been known that the sensitivity of the IVIS (trademark) Lumina Imaging System reduces at the absorption wavelength of a J-aggregate because according to the specification sheet of the system, its quantum efficiency is more than 85% at 500 to 700 nm and is more than 30% at 400 to 900 nm.

In the photoacoustic imaging method according to this embodiment, the wavelength of irradiation light may be selected depending on a laser light source to be used. In the fluorescence imaging method according to this embodiment, in order to efficiently acquire an acoustic signal, the specimen or the like is preferably irradiated with light having a wavelength of 600 nm to 1,300 nm in a near infrared region called “the optical window” where the influence of absorption and diffusion of light in a living organism is small.

Fluorescence from the contrast agent according to this embodiment can be detected and converted to an electrical signal with a fluorescence detector. Based on the electrical signal obtained with the fluorescence detector, the position or size of an absorber in the specimen or the like can be calculated. For example, when the contrast agent is detected above a threshold as a reference, a substance derived from the particle is estimated to be present in the specimen, or a substance derived from the particle can be estimated to be present in the sample obtained from the specimen.

When the contrast agent according to this embodiment is administered to the specimen, a lymph node, in particular, a sentinel lymph node which a cancer cell that has flowed from a cancer primary focus into a lymph duct reaches first can be suitably detected. In this case, a contrast agent for a lymph node is injected into a tumor or around the tumor, and the detection of the contrast agent is performed at an appropriate time after the injection.

(Photoacoustic Imaging Method)

The contrast agent according to this embodiment may be used for a photoacoustic imaging method. It should be noted that the term “photoacoustic imaging” as used herein is a concept including photoacoustic tomography (tomogram method). A photoacoustic imaging method using the contrast agent according to this embodiment includes at least the steps of: administering the contrast agent according to this embodiment to a specimen or a sample obtained from the specimen; irradiating the specimen or the sample obtained from the specimen with pulse light; and measuring a photoacoustic signal from a substance derived from the particle present in the specimen or in the sample obtained from the specimen.

An example of the photoacoustic imaging method using the contrast agent according to this embodiment is as described below. That is, the contrast agent according to this embodiment is administered to a specimen, or is added to a sample such as an organ obtained from the specimen. It should be noted that the specimen refers to all living organisms such as an experimental animal and a pet without any particular limitation. Examples of the specimen or the sample obtained from the specimen may include an organ, a tissue, a tissue section, a cell, and a cell lysate. After the administration or addition of the particle, the specimen or the like is irradiated with laser pulse light having a wavelength in a near infrared region.

In the photoacoustic imaging method according to this embodiment, the wavelength of irradiation light may be selected depending on a laser light source to be used. In the photoacoustic imaging method according to this embodiment, in order to efficiently acquire an acoustic signal, the specimen or the like is preferably irradiated with light having a wavelength of 600 nm to 1,300 nm in a near infrared region called “the optical window” where the influence of absorption and diffusion of light in a living organism is small, in particular, light having a wavelength of 700 nm to 900 nm.

A photoacoustic signal (acoustic wave) from the contrast agent according to this embodiment is detected and converted to an electrical signal with an acoustic wave detector such as a piezoelectric transducer. Based on the electrical signal obtained with the acoustic wave detector, the position or size of an absorber in the specimen or the like, or the optical characteristic value distribution of a molar absorption coefficient or the like can be calculated. For example, when the contrast agent is detected above a threshold as a reference, a substance derived from the particle is estimated to be present in the specimen, or a substance derived from the particle can be estimated to be present in the sample obtained from the specimen.

When the contrast agent according to this embodiment is administered to the specimen, a lymph node, in particular, a sentinel lymph node which a cancer cell that has flowed from a cancer primary focus into a lymph duct reaches first can be suitably detected. In this case, a contrast agent for a lymph node is injected into a tumor or around the tumor, and the detection of the contrast agent is performed at an appropriate time after the injection.

In the present invention, quenching due to the accumulation of the dye is caused by suppressing the leakage of the dye, and hence the energy of the applied pulse light is prevented from being used in fluorescent emission and can be converted into an additionally large quantity of thermal energy. Consequently, an acoustic signal can be acquired in an additionally efficient manner.

Specific reagents and reaction conditions to be used upon production of particles each containing ICG are given in the following examples. However, these reagents and reaction conditions can be modified, and such modifications are included in the scope of the present invention. Therefore, the following examples are intended to aid the understanding of the present invention and by no means limit the scope of the present invention.

### Example 1

An example of the formation of the J-aggregate of ICG and an example of a suppressing effect on the J-aggregate are described below.

FIG. 4 shows the list of the results of the measurement of the spectral shifts of aqueous solutions containing 1.5 mM of indocyanine green (ICG, manufactured by Nihon Koteisho Kyokai) and various concentrations of 100 mM or less of urea before and after heating at 60° C. for 30 minutes, the measurement being performed at the respective urea concentrations of 100 mM or less. In addition, FIG. 5 shows the list of the results of the measurement of the spectral shifts of an aqueous solution containing 1.5 mM of indocyanine green (ICG, manufactured by Nihon Koteisho Kyokai) and citric acid, and free of urea before and after heating at 60° C. for 30 minutes, the measurement being performed at respective citric acid concentrations of 100 mM or less.

FIG. 4 and FIG. 5 show the results of wavelength shifts based on additive concentration series. In FIG. 4 and FIG. 5, a square at the left end shows the absorption spectrum of the additive. The axis of abscissa of each square indicates a wavelength, and values at its left and right ends are 500 nm and 950 nm, respectively, and the axis of ordinate thereof indicates an absorbance and its scale is arbitrary. The additive in this case refers to urea.

The upper stage of the two vertical squares shows an absorption spectrum before the heating and the lower stage thereof shows an absorption spectrum after the heating.

The square at the left end corresponds to a blank free of ICG, a second square from the left end shows the absorption spectrum of a solution obtained by adding 100 mM of the additive to 1.5 mM of ICG, and the upper stage and the lower stage show spectra before and after the heating, respectively. A third square from the left end corresponds to a solution to which the additive is added at a concentration one-half as high as that of the second square. Similarly, any subsequent square shows the spectrum before or after the heating of such a solution that only the concentration of the additive is reduced by half as compared with the adjacent one while the concentration of ICG is kept at 1.5 mM.

The J-aggregate of ICG shows an absorption peak at 895 nm and hence a suppressing effect on J-aggregation can be confirmed based on the presence or absence of the peak.

In a concentration range indicated by α in FIG. 4, the J-aggregation of ICG is not suppressed because the concentration of urea is low. In addition, in a range indicated by β in FIG. 5, the J-aggregation of ICG is not suppressed because urea is not added.

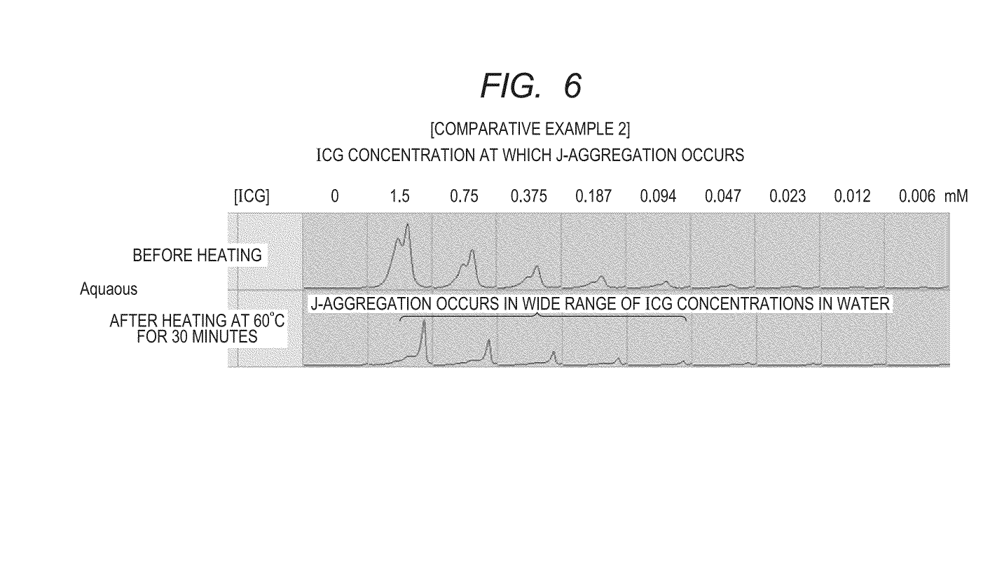

In addition, FIG. 6 shows the results of the measurement of the spectral shifts of an ICG aqueous solution before and after heating at 60° C. for 30 minutes, the measurement being performed at an ICG concentration of 1.5 mM or less for the reproduction of the condition of “J-aggregation and disaggregation of indocyanine green in water”, Chemical Physics, Volume 220, 385-392 (1997) under which ICG J-aggregates.

In FIG. 6, a square at the left end shows the absorption spectrum of only water free of ICG. A second square from the left end corresponds to a 1.5 mM ICG aqueous solution, and an upper stage and a lower stage show spectra before and after the heating, respectively. A third square corresponds to an ICG aqueous solution having a concentration one-half as high as that of the second square. Similarly, any subsequent square shows the spectrum before or after the heating of such a solution that the ICG concentration is reduced by half as compared with the adjacent one.

The axis of abscissa of each square indicates a wavelength, and values at its left and right ends are 500 nm and 950 nm, respectively, and the axis of ordinate thereof indicates an absorbance and its scale is arbitrary.

As shown in FIG. 4, when 100 mM of urea are added to the 1.5 mM ICG aqueous solution, an absorption peak wavelength does not change after the heating as compared with that before the heating and the J-aggregation is suppressed. A suppressing effect on the formation of the J-aggregate is sustained until the urea concentration is reduced to around 6.25 mM.

Although it has been simultaneously known that the H-aggregate of ICG has absorption at a wavelength around 700 nm, the addition of urea also suppresses the formation of the H-aggregate.

It should be noted that when the urea concentration reduces, ICG forms a J-aggregate after heating and the aggregate has an absorption peak at around 895 nm. Thus, a urea concentration range in which the suppression of the J-aggregation is effective has been found.

On the other hand, in FIG. 5, a J-aggregate is formed in citric acid after heating irrespective of the citric acid concentration. In addition, when the citric acid concentration is higher than 12.5 mM, agglomeration precipitation also occurs and hence the base line of a solution spectrum lowers.

In addition, in FIG. 6, the concentration of ICG in a water solvent is set to 1.5 mM or less. However, a J-aggregate can be formed even in an ICG concentration range lower than 1.5 mM experimented in, for example, “J-aggregation and disaggregation of indocyanine green in water”, Chemical Physics, Volume 220, 385-392 (1997). This example has shown that the J-aggregation of ICG is suppressed by adding urea at a concentration of 6.25 mM or more.

Although the mechanism via which the J-aggregation is suppressed is unknown, urea breaks a water cluster in sunder, which may facilitate the bonding of hydration water to ICG. It is assumed that as a result of the foregoing, the agglomeration of ICG molecules is suppressed and ICG continues to exist as a monomer even when heated, and hence no absorption wavelength shift occurs.

The concentration of the chaotropic agent of the present invention has the following feature: the concentration is 1 mM or more and 10 M or less, and the concentration of urea is particularly preferably 10 mM or more and 1 M or less.

A lower limit for the concentration is the range shown in each of FIG. 4 and FIG. 5 where the agglomeration-suppressing effect of the addition of urea appears.

Meanwhile, an upper limit range for the concentration is described below.

It has been known that the water solubility of urea is 107.9 g/100 ml (20° C.) and its 50% lethal dose LD50 is 8,500 mg/kg (oral, rat).

10.79 Grams of urea dissolve in 10 ml of an intravenous injection (the same liquid amount as that of a drug for hepatic and circulatory function tests “Diagnogreen”). The amount is 1,079 g per 1 liter and the molecular weight of urea is 60.06, and hence up to 17.9 M of urea dissolve therein.

In addition, when its 50% lethal dose is converted in terms of a body weight of 50 kg, a relationship of LD50=8.5 g/kg×50 kg=425 g is satisfied. As described above, however, only up to 10.79 g of urea dissolve in 10 ml of an injection. In addition, 600.6 g×10/1,000=6.006 g of urea are present in 10 ml of the injection to which urea is added at the upper limit for the urea concentration of the present invention, i.e., 10 M, and the ratio of the amount to the 50% lethal dose LD50 in terms of a body weight of 50 kg is 6.006 g/425 g=1.41×10⁻². Accordingly, a sufficient agglomeration-suppressing effect was obtained at a concentration equal to or less than a no-observed-effect level about one-hundredth of the 50% lethal dose LD50.

### Example 2

(Preparation and Comparison of ICG-Containing Particles in Various Systems)

ICG-containing particles of Example 3, and Comparative Examples 1 and 2 were each prepared by combining an internal aqueous phase and an external aqueous phase as shown in Table 2. It should be noted that the internal aqueous phase refers to a phase incorporated into a particle and the external aqueous phase refers to a phase outside the particle.

Example 3 shows an example in which there is a pH gradient between the external aqueous phase and the internal aqueous phase, and the external aqueous phase contains urea. Comparative Example 1 shows an example in which the pH gradient exists but the external aqueous phase does not contain urea. Comparative Example 2 shows an example in which no pH gradient exists and the external aqueous phase does not contain urea.

In each of the examples, a method of preparing an empty liposome is as described below. That is, distearoyl phosphatidylcholine (DSPC, COATSOME MC-8080, manufactured by NOF CORPORATION), cholesterol (manufactured by Wako Pure Chemical Industries, Ltd.), and distearoyl phosphatidylethanolamine-polyethylene glycol (DSPE-PEG, manufactured by NOF CORPORATION) were weighed at a weight ratio of 3:1:1. The three kinds of lipids were dissolved in a total amount of 102 mg in 2 ml of a methanol/chloroform (1:1) solution, and the mixture was stirred at 37° C. for 1 hour. After that, the solvents were removed by distillation and the residue was vacuum-dried at room temperature overnight. 10 Milliliters of each of the internal aqueous phase solutions shown in Table 2 were added to the resultant lipid dried and hardened product (per the total amount of the three kinds of lipids, i.e., 102 mg), and the mixture was stirred at 37° C. for 1 hour. Then, the mixture was subjected to an ultrasonic treatment at 60° C. for 30 minutes with a bath type ultrasonic apparatus (three-frequency ultrasonic cleaner VS-100III, manufactured by AS ONE Corporation) (the mixture was irradiated with an ultrasonic wave according to a cycle “28 kHz for 60 seconds-45 kHz for 60 seconds-100 kHz for 3 seconds”). After that, the mixture was passed through a membrane having a pore size of 0.22 μm. The external aqueous phase of each empty liposome dispersion liquid that had been passed was replaced with an internal aqueous phase solution containing 10 mM of HEPES and 150 mM of NaCl, and having a pH of 7.3 shown in Table 2 by ultrafiltration (stirring type cell, Ultrafiltration Membrane 300 KDa, manufactured by Millipore Corporation). After that, the resultant was concentrated, and the total lipid concentration of DSPC and cholesterol was adjusted to 40 mg/mL. The quantitative determination of DSPC and cholesterol was performed with a commercial determination kit (Phospholipid C-Test Wako, Cholesterol E-Test Wako, manufactured by Wako Pure Chemical Industries, Ltd.).

TABLE 2

| | Internal aqueous phase | External aqueous phase |
|---|---|---|
| Example 3 | HEPES 10 mM, | Citric acid 10 mM, urea |
| | NaCl 150 mM, | 100 mM, pH 3.0 |
| Comparative | pH 7.3 | Citric acid 10 mM, pH |
| Example 1 | | 3.0 |
| Comparative | | HEPES 10 mM, pH 7.3 |
| Example 2 | | |

The solution containing 10 mM of HEPES and 150 mM of NaCl, and having a pH of 7.3 is a buffer solution prepared by dissolving 2-[4-(2-hydroxyethyl)-1-piperazinyl]-ethanesulfonic acid (HEPES, manufactured by Life Technologies) in water at a concentration of 10 mM and adding 10 mM sodium hydroxide to adjust the pH to 7.3.

The solution containing 10 mM of citric acid and having a pH of 3.0 is a buffer solution prepared by dissolving citric acid monohydrate (manufactured by Nacalai Tesque) and trisodium citrate dihydrate (manufactured by Nacalai Tesque) in water to adjust the pH to 3.0. The solution containing 10 mM of citric acid and 100 mM of urea, and having a pH of 3.0 is a buffer solution prepared by adding 6 g of urea (manufactured by Wako Pure Chemical Industries, Ltd.) to 1 L of the citric acid buffer solution so that the concentration of urea may become 100 mM.

### Example 3

### ICG-Containing Particle Prepared with System in which there is pH Gradient Between External Aqueous Phase and Internal Aqueous Phase, and the External Aqueous Phase Contains Urea

### Example 3-1

### Preparation of ICG-Containing Particle

Indocyanine green (ICG, manufactured by Nihon Koteisho Kyokai) was dissolved in the external aqueous phase solution of Example 3 containing 10 mM of citric acid and 100 mM of urea, and having a pH of 3.0 to produce an ICG solution having an ICG concentration of 6 mg/ml. The ICG solution was added to the empty liposome prepared in Example 2 to prepare a liposome subjected to an ICG encapsulation treatment under the following conditions. That is, the ICG solution and the dispersion liquid of the empty liposome prepared in the foregoing were each placed in a thermostat at 60° C. for 15 minutes and warmed to 60° C. 2.5 Milliliters of the ICG solution were added to 2.5 mL of the empty liposome dispersion liquid, and the mixture was stirred at 60° C. for 30 minutes. After that, 15 ml of the external aqueous phase solution were added to the mixture and a liposome dispersion liquid was recovered. Further, the recovered liposome dispersion liquid was purified by subjecting the liquid to an ultrafiltration treatment (with an Ultrafiltration Membrane 300 KDa), while further injecting the external aqueous phase solution, to remove free ICG. After the purification, the liquid was concentrated to an amount of about 4 ml and recovered.

In order for the purification to be further advanced and for a component having a small particle size to be recovered, an ICG-containing particle was centrifuged with a centrifugal separator at an acceleration of 288,000 G for 20 minutes. A liposome having a large particle size and impurities that had been precipitated were removed, and only a supernatant was recovered.

### Example 3-2

### Absorption Spectrum Measurement

The ICG encapsulation-treated liposome obtained in Example 3-1 was diluted with the external aqueous phase solution of Example 3, its absorbance in the wavelength range of from 500 to 950 nm was measured, its maximum absorption wavelength and Abs895/Abs780 ratio were determined, and the formation of a J-aggregate in the particle was confirmed.

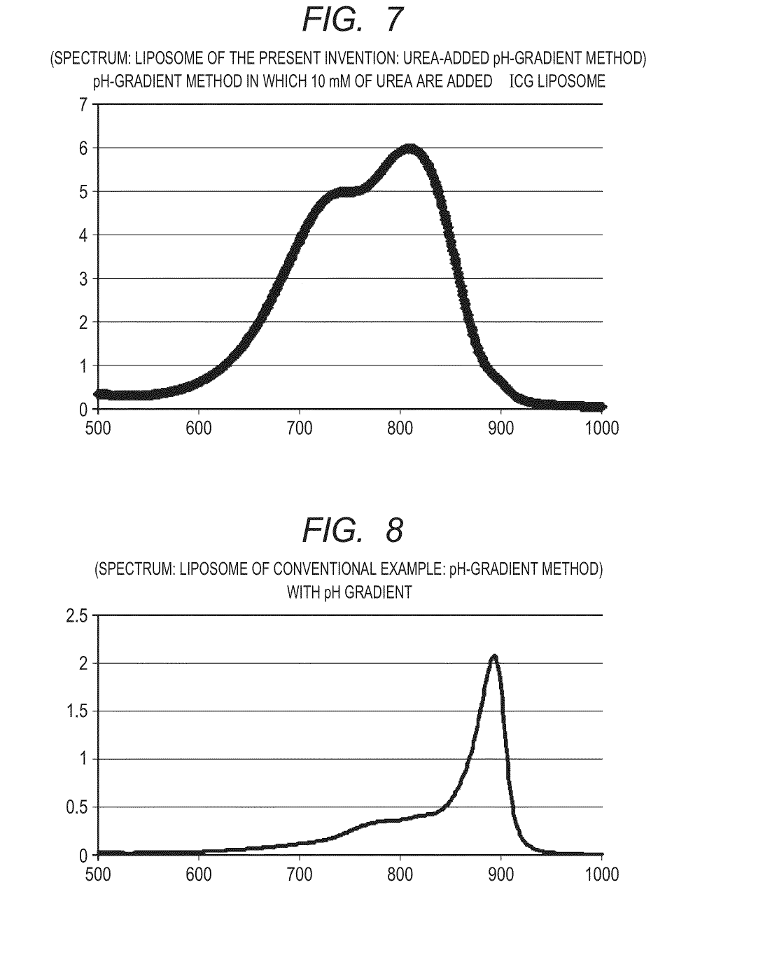

It was found (FIG. 7) that the ICG-containing particle of this example, i.e., the liposome provided with a pH gradient, and prepared by using a citric acid buffer solution and urea as an external aqueous phase had an Abs895/Abs780 ratio as low as 0.2 or less, and the particle did not show a maximum absorption wavelength around 895 nm and hence nearly no ICG in the particle formed a J-aggregate.

Dextran 40 or sodium chloride (NaCl) may be further added to the internal aqueous phase.

### Example 3-3

### Particle Size Measurement

The particle size of the ICG-containing particle obtained in Example 3-1 was measured by DLS (Zeta-Sizer Nano, manufactured by Malvern Instruments).

FIG. 10 shows the particle sizes of an empty liposome 1 in the present invention to which a pH-gradient method is applied, and a liposome after encapsulation and purification. It was found that the ICG-containing particle of the present invention was able to be prepared as a particle having a particle size around 100 nm.

It should be noted that the ICG-containing particle has an average particle size and particle size distribution slightly smaller than those of the empty liposome because the liposome having an increased particle size and the impurities have been removed by centrifugation in order that a component having a small particle size may be recovered.

### Comparative Example 1

### ICG-Containing Particle Prepared with System in which there is pH Gradient Between External Aqueous Phase and Internal Aqueous Phase, but the External Aqueous Phase is Free of Urea

### Comparative Example 1-1

### Preparation of ICG-Containing Particle

An ICG-containing particle was prepared by the same treatment as that of Example 3-1 except that the external aqueous phase for Comparative Example 1 shown in Table 2 was used as an external aqueous phase.

(Absorption Spectrum Measurement)

The absorbances at 780 nm and 895 nm of the resultant ICG-containing particle were measured. Table 5 shows the Abs895 and the Abs780, and an Abs895/Abs780 ratio.

As shown in FIG. 8, the liposome in this comparative example provided with a pH gradient and prepared by using only a citric acid buffer solution as an external aqueous phase without incorporating urea had an Abs895/Abs780 ratio as high as 2 or more, and showed a local maximum of an absorbance between 880 nm and 910 nm. Accordingly, it was found that a liposome encapsulating J-aggregated ICG was prepared.

### Comparative Example 2

### ICG-Containing Particle Prepared with System in which there is No pH Gradient Between External Aqueous Phase and Internal Aqueous Phase, and the External Aqueous Phase is Free of Urea

### Comparative Example 2-1

### Preparation of ICG-Containing Particle

An ICG-containing particle was prepared by the same treatment as that of Example 3-1 except that the external aqueous phase for Comparative Example 2 shown in Table 2 was used as an external aqueous phase.

(Absorption Spectrum Measurement)

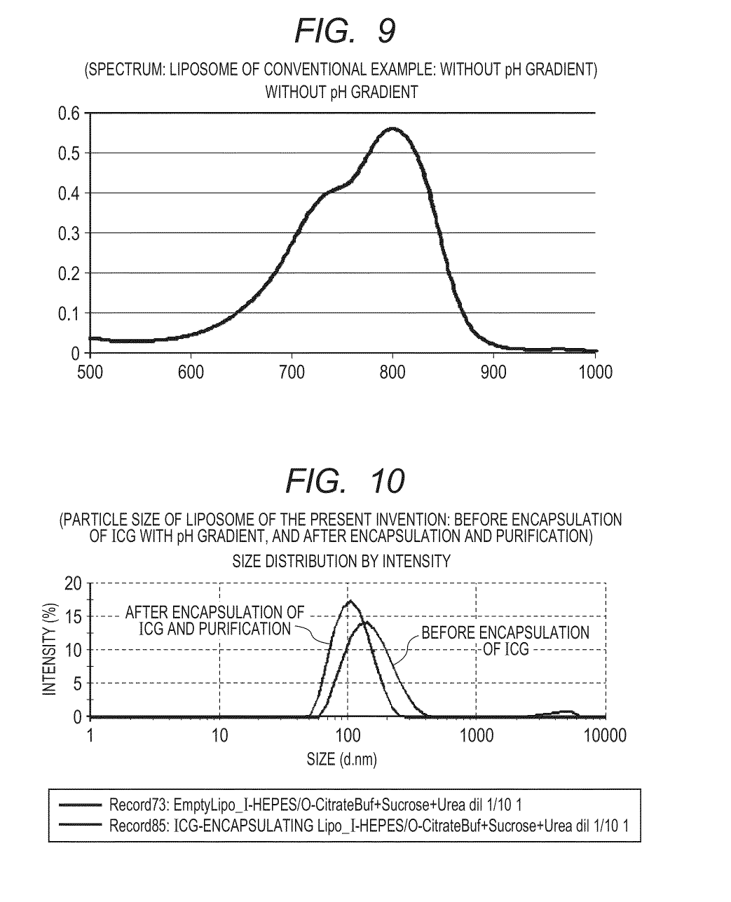

It was found (FIG. 9) that as in the present invention shown in FIG. 7, the particle prepared in this comparative example had an Abs895/Abs780 ratio as low as 0.2 or less, and the particle did not show a maximum absorption wavelength around 895 nm and hence nearly no ICG in the liposome formed a J-aggregate.

Table 3 shows comparison between the physical property values of the ICG-containing particle when a pH gradient is provided and when the pH gradient is not provided.

As shown in the ICG recovery ratio and content without any pH gradient in Table 3, when the pH gradient is not provided, the particle has a low ICG encapsulation ratio. It is apparent that the pH gradient needs to be provided for increasing the ICG content.

In the example of Table 3, a concentrating effect obtained with the pH gradient is 5.7 to 6.0 times as high as that obtained without any pH gradient.

TABLE 3

| | With pH | Without pH | |
|---|---|---|---|
| | gradient | gradient | Effect |
| | (Comparative | (Comparative | of pH |
| Sample No. | Example 2) | Example 3) | gradient |
| Internal aqueous phase | HEPES + Nacl 150 mM | HEPES + Nacl 150 mM | |
| External aqueous phase | Citric acid | HEPES | |
| Concentration of ICG loaded into | 6 mg/mL | 6 mg/mL | |
| external aqueous phase | | | |
| Particle size after encapsulation | 107 | 105 | |
| and Extr·UF filtration (nm) | | | |
| Dry weight (mg/mL) | 30.1 | 28.6 | |
| ICG concentration (mg/mL) | 2.056 | 0.345 | |
| Loading amount of ICG (mg) | 15 | 15 | |
| ICG recovery ratio (%) | 27.4 | 4.6 | 6.0 |
| ICG content (%) | 6.83 | 1.21 | 5.7 |
| Number of particles | 4.70E+13 | 4.72E+13 | |
| Maximum absorption wavelength | 893 | 804 | |

### Example 3

### Measurement of Photoacoustic Signal

The intensity of the photoacoustic signal of the ICG-containing particle obtained in Example 2 was measured. As a comparative example, an ICG aqueous solution was similarly subjected to the measurement. The measurement of the photoacoustic signal was performed by: irradiating the sample with pulse laser light; detecting the photoacoustic signal from the sample with a piezoelectric element; amplifying the signal with a high-speed preamplifier; and acquiring the amplified signal with a digital oscilloscope. Specific conditions for the measurement are as described below. Titanium sapphire laser (manufactured by Lotis Ltd.) was used as a light source. The conditions of a wavelength of 780 nm or 895 nm, an energy density of 12 mJ/cm², a pulse width of 20 nanoseconds, and a pulse repetition of 10 Hz were adopted. A Model V303 (manufactured by Panametrics-NDT) was used as an ultrasonic transducer. The conditions of a central band of 1 MHz, an element size of φ0.5, a measurement distance of 25 mm (non-focus), and an amplification of +30 dB (Ultrasonic Preamplifier Model 5682 manufactured by Olympus Corporation) were adopted. A measurement vessel was a cuvette made of polystyrene, and had an optical path length of 0.1 cm and a sample volume of about 200 μl. Water was used as a solvent. A DPO04104 (manufactured by TEKTRONIX, INC.) was used as a measuring device, and the measurement was performed under the conditions of: trigger: detection of photoacoustic light with a photodiode; and data acquisition: 128 times (128 pulses) on average.

As a result of the measurement of the photoacoustic signal at a wavelength of 780 nm, the ICG-containing particle of Example 3 was found to generate a photoacoustic signal having an intensity 1.7 times as high as that of the ICG aqueous solution.

The particle according to the present invention can stably hold ICG in itself without causing the J-aggregation of ICG.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-105265, filed May 17, 2013, which is hereby incorporated by reference herein in its entirety.

What is claimed is:

1. A method of producing an indocyanine green-containing particle, comprising mixing indocyanine green and a particle in a solution containing 1 mM or more and 10 M or less of a chaotropic agent.

2. A method of producing an indocyanine green-containing particle according to claim 1, wherein the solution containing the chaotropic agent contains 6.25 mM or more of the chaotropic agent.

3. A method of producing an indocyanine green-containing particle according to claim 1, wherein the solution containing the chaotropic agent contains 100 mM or less of the chaotropic agent.

4. A method of producing an indocyanine green-containing particle according to claim 1, wherein in the mixing of indocyanine green and the particle in the solution containing 1 mM or more and 10 M or less of the chaotropic agent, a solution containing the chaotropic agent in which indocyanine green is dissolved and the particle are mixed.

5. A method of producing an indocyanine green-containing particle according to claim 1, wherein the chaotropic agent comprises one of urea, guanidine, iodine, and ions thereof.

6. A method of producing an indocyanine green-containing particle according to claim 1, wherein the chaotropic agent comprises urea.

7. A method of producing an indocyanine green-containing particle according to claim 1, wherein the particle comprises a liposome.

8. A method of producing an indocyanine green-containing particle according to claim 1, wherein the solution containing the chaotropic agent has a pH of less than 7.

9. An indocyanine green-containing particle, comprising:
indocyanine green; and
a particle,
wherein the indocyanine green-containing particle further comprises a chaotropic agent.

10. An indocyanine green-containing particle according to claim 9, wherein the indocyanine green-containing particle has an average particle size of 1,000 nm or less.

11. An indocyanine green-containing particle according to claim 9, wherein the indocyanine green-containing particle has an average particle size of 200 nm or less.

12. An indocyanine green-containing particle according to claim 9, wherein the particle contains a phospholipid.

13. An indocyanine green-containing particle according to claim 9, wherein the particle comprises a liposome.

14. An indocyanine green-containing particle according to claim 9, wherein the indocyanine green-containing particle further comprises cholesterol.

15. A photoacoustic imaging contrast agent, comprising the indocyanine green-containing particle according to claim 9.

16. A contrast agent for photoimaging, comprising:
the indocyanine green-containing particle according to claim 9; and
a dispersion medium for dispersing the indocyanine green-containing particle.
